(19) United States
(12) Patent Application Publication
(10) Pub. No.: US 2005/0137548 A1
(43) Pub. Date: Jun. 23, 2005

(54) Sanitary panties with pocket for wipes and spare panties

(76) Inventor: Francine Riley, Jonesboro, GA (US)

Correspondence Address: Donald W. Meeker;Patent Agent, 924 East Ocean Front #E, Newport Beach, CA, 92661, US

(21) Appl. No.: 10/742,261

(22) Filed: Dec. 20, 2003

(51) Int. Cl. A61F 13/15; A61F 13/20

(52) U.S. Cl. 604/385.01; 604/385.11

(57) ABSTRACT

A disposable pair of camouflaged sanitary panties with a built-in sanitary napkin, side fasteners, and an internal pocket for storage of cleansing wipes and spare sanitary panties, which may be used by women in outdoor situations that require the woman to quickly change underwear in a concealed manner.

### BACKGROUND OF THE INVENTION

1. Field of the Invention

The present invention relates to disposable absorbent side fastening underwear and particularly to a pair of camouflaged panties with a built in sanitary napkin, side fasteners, and an internal pocket for storage of cleansing wipes and spare panties, which may be used by women in situations such as combat or hunting that require the woman to quickly change underwear in a concealed manner.

2. Description of the Prior Art

An increasing number of women are spending a greater amount of time outdoors. This is due to a number of reasons, including involvement in the military and in such sports as hunting and fishing, and also due to time spent in outdoor jobs such as construction. Women in these situations have increasingly realized that toilet facilities can be absent when needed. This problem is sometimes exacerbated, particularly in the military, sporting, or construction environments, by the likelihood that far more men will be present than women.

On the combat field all soldiers are required to keep one hand on their weapons at all times. This presents a problem for women who need to change sanitary napkins during a combat situation.

Changing sanitary pads quickly with full uniform including pants and boots and equipment necessitates that the operation may be performed without removal of the pants and boots.

Furthermore outdoor conditions expose women to unsanitary conditions and often the women's hands are soiled from outdoor activity.

In combat situations, it is desirable not to reveal the presence of women in combat, so that disposing of sanitary pads is a problem.

Most of these problems have not been adequately addressed by the inventions in the prior patents.

Prior art U.S. Pat. No. 6,475,204, issued Nov. 5, 2002 to Walker, indicates a diaper construction wherein one or more baby wipes is included in the diaper so that the child care giver always has a baby wipe to change the diaper. To prevent removal of the baby wipes by the baby, the baby wipe is contained within a cavity of a sealed pocket member provided within the diaper that is accessible only by tearing an access opening through an outer layer of the diaper and then rupturing a wall of the pocket member to gain access to the baby wipe.

Prior art U.S. Pat. No. 5,241,710, issued Sep. 7, 1993 to Lockhart, provides a two piece sanitary panty with one of the pieces being a removable lower or crotch portion. This portion has an inner section designed for the absorption of menses, and an outer moisture resistant backing that includes both a front and rear engaging means and a pocket adapted to hold a pre-wrapped moistened towelette. Thus, when the inner section becomes soiled, it is easily replaceable by disengaging the crotch portion and replacing it with a new one.

Prior art U.S. Pat. No. 5,944,708, issued Aug. 31, 1999 to Philpott, concerns a protective menstrual panty, which comprises a body portion worn around a lower torso of a female person during menses. A liquid-proof elastic crotch portion is provided. A facility is for securing opposite ends of the crotch portion between front and rear lower segments of the body portion, so as to form two openings for insertion of one of a pair of legs of the female person through each opening. A structure is for holding a sanitary napkin within the crotch portion in a proper position, so that the sanitary napkin will fit snugly against the inner thighs of the legs, and not tilt to leak from the sides thereof.

Prior art U.S. Pat. No. 6,041,445, issued Mar. 28, 2000 to Davitt, describes an adaptive undergarment for persons with specialized urological needs. The adaptive undergarment provides an elongate pocket for storing specialized urological articles. The elongate pocket is located preferably in a parallel spaced relationship with the waistband of the user's undergarment. The adaptive undergarment may have an additional pocket elsewhere on the undergarment for storing items such as antibacterial wipes. Each pocket is open at one edge to receive and provide access to the stored items. Any type of undergarment conforming to any user's specialized urological needs is adaptable into this adaptive undergarment.

Prior art U.S. Pat. No. 5,261,531, issued Nov. 16, 1993 to Nieves, puts forth a feminine hygiene package, which comprises an inexpensive and compact sanitary kit for cleansing the body after discharge of bodily fluids. A dry wipe, an enclosed wet wipe, and a sanitary napkin are enclosed in an enclosure protecting the components during storage. The enclosure has length and width not exceeding 20 cm and 10 cm, respectively, and is usable in disposing of the wipes and of the napkin, if desired, after use.

Prior art U.S. Pat. No. 6,308,340, issued Oct. 30, 2001 to Cook, discloses an inside pocket formed in a pair of underpants by attaching an inside panel to the front portion of the underpants. The pocket is open at the top to receive a spare sanitary napkin, which is carried horizontally in the pocket during the menses.

Prior art U.S. patent application No. 20030136704, published Jul. 24, 2003 by Burgess, indicates a girl's growing up kit. A sanitary napkin package and kit is provided for emergency use during a first, unexpected, or emergency onset of a menstrual period or menses, which contains a sanitary napkin located inside a compartmentalized container; a plurality of pockets attached to the interior surface of said container, each of said pockets having a discrete hygienic item therein; one of said hygienic items being a substantially hour-glass shaped moisture barrier having an upper surface and a lower surface, an adhesive being at the edge of the upper surface, and an absorbent material attached to the upper surface and surrounded by the adhesive; the lower surface having a pattern such as a shade of khaki, denim or the like thereon whereby the pattern camouflages the presence of the shaped moisture barrier when attached to the crotch of pants on the outside thereof or when attached to the user's menstrually soiled exposed undergarments during an emergency.

Prior art U.S. patent application No. 20030065301, published Apr. 3, 2003 by Elliott, concerns a disposable diaper that contains or has attached wipe(s). The wipe(s) will be one or more individual pieces of wet/dry/fragrance/lotion material. The material may be cloth, tissue, napkin, towel, towelette, sponge, or a handi-wipe.

Prior art U.S. Pat. No. 4,940,463, issued Jul. 10, 1990 to Leathers, claims a disposable combined panty with sanitary napkin. The disposable panty has a tubular body portion with an hourglass configuration to provide an intermediate crotch portion and opposing front and rear waist portions. A sanitary napkin is disposed within the crotch portion between the outer and inner layers of the body portion. A ribbon member passes through the front and rear waist portions to secure the pantry to the wearer, where the ends of the ribbon member are removably secured together. The sanitary napkin includes a frame member provided with absorbent balls disposed in the opening of the frame member to increase the absorption of the menstrual discharge. A flexible liquid impervious material is disposed between the outer layer of the body portion and the sanitary napkin to prevent the passage of the menstrual discharge to the outer layer. The panty with the sanitary napkin can be folded upon itself to function solely as a sanitary napkin.

Prior art U.S. Pat. No. 6,367,089, issued Apr. 9, 2002 to Van Gompel, puts forth a three dimensional disposable menstrual panty for holding a sanitary pad. The panty may have elasticized leg and waist openings and be stretchable about the hip and stomach regions of a user. The panty provides backup leakage protection to the sanitary pad. The panty includes an absorbent barrier composite positioned in the crotch area and extending into the body of the disposable panty front and back and over the leg elastics to trap pad leakage inside the pant and prevent liquid strike through onto outer clothing and bed linen.

Prior art U.S. Pat. No. 6,240,569, issued Jun. 5, 2001 to Van Gompel, concerns a three dimensional disposable menstrual panty for holding a sanitary pad. The panty may have elasticized leg and waist openings and be stretchable about the hip and stomach regions of a user. The panty provides backup leakage protection to the sanitary pad. The panty includes an absorbent barrier composite positioned in the crotch area and extending into the body of the disposable panty front and back and over the leg elastics to trap pad leakage inside the pant and prevent liquid strike through onto outer clothing and bed linen.

Prior art U.S. Pat. No. 5,441,493, issued Aug. 15, 1995 to Gonzalez-Anguiano, describes disposable underpants that include an underpant body having an inner surface and an outer surface, a longitudinal undercrotch portion, and front and rear end portions positioned at opposing ends of the longitudinal undercrotch portion and transverse thereto. A sanitary napkin member is positioned over the longitudinal undercrotch portion and extends substantially a length of the longitudinal undercrotch portion, the sanitary napkin including front, rear, and longitudinal edges. The sanitary napkin member is secured to the longitudinal undercrotch portion of the underpant body on the inner surface thereof and only along the rear and longitudinal edges of the sanitary napkin. An increased density of material is provided within the underpant body at the rear end portion thereof and coextensive with the longitudinal undercrotch portion, thus resisting tearing of the rear end portion during flexing of the disposable underpants.

Prior art U.S. Pat. No. 5,711,034, issued Jan. 27, 1998 to Cillik, discloses menstrual underpants that comprise a garment configured with a front portion, a rear portion, a crotch area, having two leg openings and a waist opening, so as to be worn about a lower torso of a woman. Provision is made for engaging opposite ends of an absorbent pad. A facility is for securing the engaging structure to the crotch area of the garment. The absorbent pad will be held in place at the crotch area and not move up and down, while edges of the absorbent pad will not curl up, to allow the woman to have more comfort, when wearing the garment.

Prior art U.S. Pat. No. 5,325,543, issued Jul. 5, 1994 to Allen, provides a disposable undergarment for releasably securing an absorbent pad in a pocket located in the crotch portion of the undergarment. A method for making the same is also disclosed. The undergarment is made with a body portion and pocket portion. The pocket portion overlays the crotch portion and is attached to the body portion along a sufficient portion of its peripheral edge to form a pocket therebetween. The pocket is sufficient in size to secure or store an absorbent pad or the like. Preferably, the undergarment is made of an inexpensive material so that it may be disposable.

Prior art U.S. Pat. No. 4,690,681, issued Sep. 1, 1987 to Haunschild, illustrates a disposable leakproof panty-like garment containing an integral menstrual pad. The absorbent pad portion extends from the crotch region up in both the back and the front to a point higher than normal menstrual pads. The pad extends at least up to the area where the crack between the gluteus maximus ends. Further there is an impervious member that is outside of the absorbent pad and greater in area than the absorbent pad.

Prior art U.S. Pat. No. D341,199, issued Nov. 9, 1993 to Huguley, is for the ornamental design for a disposable brief with a built-in sanitary napkin.

Prior art U.S. Pat. No. D330,590, issued Oct. 27, 1992 to Pressley, depicts the ornamental design for a disposable panty napkin.

Prior art U.S. patent application No. 20030100873, published May 29, 2003 by Hermansson, describes a disposable absorbent pant diaper with a longitudinal direction and a transverse direction, an inner cover layer, an outer cover layer, and an absorption body enclosed between the two cover layers, and having two end edges extending in the transverse direction forming a waist opening and two side edges extending in the longitudinal direction forming leg openings, the pant diaper also having a front portion and a rear portion which are connected by an intermediate crotch portion.

Prior art U.S. patent application No. 20030149418, published Aug. 7, 2003 by Katz, discloses a diaper for quick changing in a confined space, which is pre-formed with a single leg opening and an opposite open side seam. The open side seam has cooperating fastener elements to fit and secure the diaper about an infant.

Prior art U.S. Pat. No. 5,569,230, issued Oct. 29, 1996 to Fisher, indicates an individually packaged sanitary napkin having a cleansing wipe packaged therewith. The individually packaged sanitary napkin includes a wrapper that covers the sanitary napkin's adhesive fastener prior to use. The sanitary napkin wrapper can be provided with a flap or pouch for securing the used sanitary napkin for disposal. A number of alternative arrangements are disclosed for packaging the cleansing wipe with the wrapped sanitary napkin.

Prior art U.S. Pat. No. 4,560,381, issued Dec. 24, 1985 to Southwell, is for a protective, lightweight, disposable woman's hygienic panty for removeably carrying a feminine napkin during a woman's menstrual cycle or period. A relatively thin, lightweight, disposable, biodegradable, mesh-like outer panty shell is affixed to or contiguous with a relatively thick inner layer of absorbent material operably disposed about the inner surface of a lower body portion proximate at least the lower one half of the panty. Alternately, an inner shell or liner of relatively thick absorbent material having a pad-receiving depression can be fitted, temporarily or permanently, into ordinary menstrual panties or common panties or briefs. An elongated, nearly rectangular depression or indentation having oval end portions is included or formed in the relatively thick layer for carrying, aligning, or positioning a feminine napkin. The feminine napkin-shaped depression carries or positions the feminine napkin for use during the menstrual period and provides for easy removal when the napkin is soiled. The panty or liner may be discarded after a single use such as after a heavy discharge or several lightly soiled feminine napkins may be discarded before the entire panty or liner is thrown away. The invention contemplates a waist adjustable version and a one-size-fits-all version utilizing side slits and fasteners along the slits to enable substantially all sizes of women to use a single protective panty in accordance with the present invention.

Prior art U.S. Pat. No. 6,393,621, issued May 28, 2002 to Redwine, shows an undergarment that has a close body fit for use with an absorbent article, which includes an elasticized waistband, a front region, a rear region, a crotch region and a pair of elasticized leg openings. The front and rear regions are preferably elastically extensible in both the longitudinal and lateral directions. The crotch region is disposed between the front and rear regions and has greater resistance to stretching in the longitudinal direction than the front and rear regions. The crotch region is further provided with a longitudinal stretch control member that is disposed along the longitudinal centerline of the undergarment and a plurality of angled stretch control members that extend from the longitudinal stretch control member to the leg elastics at an acute angle to the longitudinal stretch control member. The stretch control members limit the stretch of the crotch region in both the longitudinal and the lateral directions that causes the crotch region to conform to a wearer's skin surface. The rear region is provided with a lifting member that cooperates with the rear region, the front region and the longitudinal stretch control member to provide a “z-direction” biasing force along the longitudinal stretch control member. This biasing force, which is greater than the body contact force in adjacent portions of the crotch region, causes the crotch region, and any absorbent article that may be disposed thereon, to be lifted into close bodily contact when the undergarment is worn.

Prior art U.S. Pat. No. 3,424,162, issued Jan. 28, 1969 to Parravicini, provides disposable hygienic underpants, which are characterized by the feature that they comprise a body portion that is at least partially of cellulose material, which is fleece-like in form. The underpants may further comprise a gauze insert or may be completely lined with gauze. An elastic band and a fabric band may be sewn or otherwise affixed to the waist and leg portions. The cellulose material may be reinforced by a number of parallel threads.

Prior art U.S. Pat. No. 3,599,638, issued Aug. 17, 1971 to Rickard, shows a disposable panty with an improved crotch construction. The panty has at least one auxiliary layer of absorbent material disposed over the crotch area, with the auxiliary layer secured to the body portion of the panty at the opposite edges of the crotch formed by the leg openings so that the auxiliary layer cooperates with the crotch area to form a receptacle for a sanitary napkin. In the preferred construction, two auxiliary layers are provided so that the auxiliary layer on the soiled side of a first napkin can be removed with the napkin, and the next napkin inserted between the crotch area and the remaining auxiliary layer.

Prior art U.S. Pat. No. 2,748,772, issued Jun. 5, 1956 to Titone, claims a disposable combined panty and sanitary napkin. The device has a body portion formed as a panty and waist and leg openings, said leg openings defining a crotch area. A sanitary napkin is supported on the crotch area and is wholly contained by an inner wall that cooperates with the crotch area to define a pocket.

Prior art U.S. patent application No. 20030114818, published Jun. 19, 2003 by Benecke, illustrates a color masking component for use with feminine sanitary pad and the like. The invention relates generally to products for absorbing bodily fluids, such as feminine sanitary pads, tampons, wound dressings, bandages, and the like. The absorbent personal article of the invention includes a color masking layer with fluid impermeable areas disposed on a fluid permeable support fabric in spaced relationship. The article may include an absorbent core, a topsheet, a backsheet, and a spreading layer in addition to or in combination with the color masking layer.

What is needed is a camouflaged disposable sanitary panty with a built-in absorbent pad and side fasteners as well as storage of spare sanitary panties and cleaning and antiseptic wipes.

### SUMMARY OF THE INVENTION

An object of the present invention is to provide a disposable sanitary panty with a built-in absorbent pad and side fasteners as well as storage of spare sanitary panties and cleaning and antiseptic wipes to enable a quick change in the field under sanitary cleaning conditions without necessitating removal of pants and boots.

Another object of the present invention is to provide camouflaged disposable sanitary panties to enable concealed disposal of the soiled panties to prevent detection by enemy forces in combat situations.

One more object of the present invention is to provide two wet wipe disposable napkins, one to clean the hands before the sanitary panty changing operation and one to clean the hands after the sanitary panty changing operation.

An additional object of the present invention is to provide one antiseptic cleaning wipe to wipe the genital area after removal of the soiled sanitary panties.

In brief, a disposable sanitary panty is provided for use in outdoor situations that allows changing without the removal of pants and footwear, which comprises a panty body with interconnectable side panels with mating hook and loop fasteners. The fasteners enable removal of the panty body by disconnecting the side panels and slipping the panty body out from the crotch and installation of the panty body by inserting the panty body under the crotch and interconnecting the side panels. The disposable sanitary panty also comprises a built-in absorbent pad in a crotch area of the panty body, which is adapted for use as a sanitary pad. The panty body is fabricated of a camouflaged material to conceal a partially undressed woman in a combat or outdoor situation and to enable concealed disposal of a soiled disposable sanitary panty to prevent detection by enemy forces.

The disposable sanitary panty also includes a pocket built into the panty body, which is adapted to provide a storage area. The storage area is adapted to hold a spare pair of disposable sanitary panties, two disposable cleaning wipes for cleaning hands, and one antiseptic disposable cleaning wipe. The two wet wipe disposable napkins allow a woman to use one wipe to clean her hands before a sanitary panty changing operation and one wipe to clean her hands after the changing operation. The antiseptic cleaning wipe is provided so that a woman may clean her genital area after removal of the soiled sanitary panties. The spare sanitary panties and cleaning and antiseptic wipes enable a quick change in the field under sanitary cleaning conditions without necessitating removal of pants and boots.

An advantage of the present invention is that it is disposable.

Another advantage of the present invention is that it provides a spare pair of disposable panties.

An additional advantage of the present invention is that it provides a way to clean the wearer's hands and genital area.

One more advantage is that the panties are camouflaged.

Yet another advantage is that the present invention may be disposed of in a concealed manner.

Still another advantage is that the panties may be quickly changed without the removal of pants or boots.

Another corollary advantage is that the panties may be changed one handedly.

### BRIEF DESCRIPTION OF THE DRAWINGS

These and other details of my invention will be described in connection with the accompanying drawings, which are furnished only by way of illustration and not in limitation of the invention, and in which drawings:

FIG. 1 is a perspective view of the disposable sanitary panties of the present invention showing the built-in pad.

### BEST MODE FOR CARRYING OUT THE INVENTION

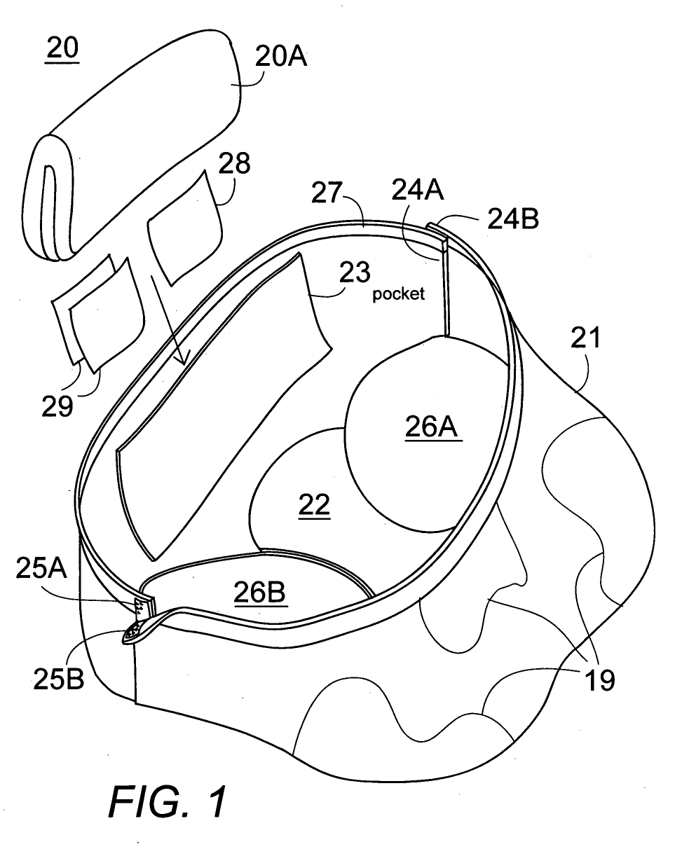

In FIG. 1, a disposable sanitary panty 20 for use in outdoor situations for changing without removing pants and footwear, which comprises a panty body 21 having interconnectable side panels 24A and 24B having removable interconnecting means for securing the side panels 24A and 24B together. The interconnecting means preferably comprises mating hook and loop fasteners 25A and 25B attached to the side panels 24A and 24B, but may employ snaps or other open and closable fasteners. The fasteners enable removal of the sanitary panty 20 from a woman by disconnecting the side panels 24A and 24B and slipping the sanitary panty 20 out from the crotch and installation of the sanitary panty 20 by inserting the sanitary panty 20 under the crotch and interconnecting the side panels 24A and 24B with outer pants pulled down but not taken off. The panty body 21 is preferably fabricated of a camouflaged material 19 to enable concealed disposal of a soiled disposable sanitary panty 20 to prevent detection by enemy forces in combat situations.

The disposable sanitary panty 20 also comprises a built-in absorbent pad 22 in the crotch area of the panty body 21, the absorbent pad 22 adapted for use as a sanitary pad.

The disposable sanitary panty 20 further comprises a pocket 23 built into the panty body 21, which is adapted for use as a storage area 23 preferably in the front for easy access. The storage area 23 is adapted for storing a spare pair of disposable sanitary panties 20A, two disposable wet cleaning wipes 29 for cleaning hands, and one antiseptic disposable cleaning wipe 28. The two wet wipe disposable wipes 29 allow a woman to use one wipe 29 to clean her hands before a sanitary panty 20 changing operation and one wipe 29 to clean the hands after a sanitary panty 20 changing operation. The antiseptic cleaning wipe 28 is provided so that a woman may clean her genital area after removal of the soiled sanitary panties 20. The spare sanitary panties 20A and cleaning and antiseptic wipes 29 and 28 enable a quick change in the field under sanitary cleaning conditions without necessitating removal of pants and boots.

The sanitary panty 20 is worn with the pocket 23 preferably located in the front near the woman's abdomen, the right leg in one leg opening 26A, the left leg in a second leg opening 26B and the pad 22 centered in contact with the genital area. Preferably an elastic waistband 27 holds the sanitary panties 20 up in a conventional manner.

In practice, a woman in combat, hunting, hiking or other outdoor activity may quickly change her disposable sanitary panties 20 in the field under sanitary cleaning conditions without removal of her pants or footwear. The woman would first remove a wet disposable cleaning wipe 29 from the pocket 23 and use it to clean her hands before the sanitary panty 20 changing operation. The woman would then remove the spare pair of panties 20A, the antiseptic wipe 28 and the remaining cleaning wipe 29 from the pocket 23 and place them in a readily accessible location. The soiled panty 20 would then be removed by disconnecting the hook and loop fasteners 25A and 25B located at the side panels 24A and 24B and slipping the soiled sanitary panty 20 out from the crotch. The antiseptic cleaning wipe 28 would then be used to clean the genital area after removal of the soiled sanitary panties 20. The spare pair of disposable sanitary panties 20A would be put on by inserting the sanitary panties 20A under the crotch and adjusting the spare sanitary panties 20A in the proper wearing position. The spare panty 20A is fastened in place by interconnecting the hook and loop fasteners 25A and 25B of the side panels 24A and 24B. The second cleaning wipe 29 is then used to clean the hands after the changing operation. The soiled disposable sanitary panty 20 may be disposed of in a concealed manner, thereby preventing detection by enemy forces in combat situations.

It is understood that the preceding description is given merely by way of illustration and not in limitation of the invention and that various modifications may be made thereto without departing from the spirit of the invention as claimed.

1. A disposable sanitary panty for use in outdoor situations for changing without removing pants and footwear, the device comprising:
a panty body having interconnectable side panels having removable interconnecting means for securing the side panels together, enabling removal of the panty body from a woman by disconnecting the side panels and slipping the panty body out from the crotch and installation of the panty body by inserting the panty body under the crotch and interconnecting the side panels;
a built-in absorbent pad in a crotch area of the panty body, the absorbent pad adapted for use as a sanitary pad;
a storage area built into the panty body, the storage area adapted for storing a spare pair of disposable sanitary panties and at least one disposable cleaning wipe for cleaning hands and at least one antiseptic disposable cleaning wipe to enable a quick change in the field under sanitary cleaning conditions without necessitating removal of pants and boots.

2. The disposable sanitary panty of claim 1 wherein the panty body in fabricated of a camouflaged material to enable concealed disposal of a soiled disposable sanitary panty to prevent detection by enemy forces in combat situations.

3. The disposable sanitary panty of claim 1 further comprising two wet wipe disposable napkins, one to clean the hands before a sanitary panty changing operation and one to clean the hands after a sanitary panty changing operation.

4. The disposable sanitary panty of claim 1 further comprising at least one antiseptic cleaning wipe to wipe a genital area after removal of the soiled sanitary panties

5. The disposable sanitary panty of claim 1 wherein the storage area comprises at least one pocket built into the panty body.

6. The disposable sanitary panty of claim 1 wherein the interconnecting means for the side panels comprises mating hook and loop fasteners attached to the side panels.
